(19) United States
(12) Patent Application Publication
(10) Pub. No.: US 2026/0117170 A1
(43) Pub. Date: Apr. 30, 2026

(54) MICROORGANISMS FOR REDUCING ODOR IN ORGANIC WASTE

(71) Applicant: MICROBIAL DISCOVERY GROUP, LLC, Franklin, WI (US)

(72) Inventors: Vanessa CHAPA, Franklin, WI (US); Megan DUERSTELER, Franklin, WI (US); Sona SON, Franklin, WI (US); Michael R. KING, Oak Creek, WI (US)

(21) Appl. No.: 19/370,293

(22) Filed: Oct. 27, 2025

Related U.S. Application Data
(60) Provisional application No. 63/714,017, filed on Oct. 30, 2024.

(51) Int. Cl. C12N 1/20; B09B 3/60; C02F 3/34; C02F 101/30; C12N 9/18; C12N 9/26; C12N 9/42; C12N 9/50; C12R 1/125

(52) U.S. Cl. CPC C12N 1/20; B09B 3/60; C02F 3/341; C02F 3/342; C12N 9/18; C12N 9/2411; C12N 9/2437; C12N 9/50; C02F 2101/30; C02F 2305/04; C12R 2001/125

(57) ABSTRACT

The present disclosure relates to composition and methods of treating waste for the purposes of, including but not limited to, degrading waste, bioremediation of waste, reducing odor in waste, reducing organics in waste, and combinations thereof. More particularly, the present disclosure relates to the use of isolated Bacillus strains Bp D10, Bs E12, and strains having all of the identifying characteristics of those strains, and combinations thereof, for treating waste.

### CROSS-REFERENCE TO RELATED APPLICATIONS

This application claims the benefit under 35 USC § 119(e) of U.S. Provisional Application Ser. No. 63/714,017, filed on Oct. 30, 2024, the entire disclosure of which is incorporated herein by reference.

### BACKGROUND

The breakdown of organic waste can result in the production of a variety of byproducts that cause nuisance odors. Odors caused by both public and private industrial water and waste treatment facilities can result in fines, complaints, and loss of business. Some odor causing byproducts of organic waste digestion can also be hazardous to human health, such as hydrogen sulfide and ammonia. Additionally, many industrial and consumer cleaning products are used to control nuisance odors in applications such as septic systems, portable toilets, grease traps, drains, and tile and surface cleaners.

Microorganisms can be an effective solution for controlling many different types of odors as they can utilize odor-causing compounds for energy, respiration, and cell growth. Bacteria use volatile fatty acids which can be broken down to generate energy and support increased cell growth. They also need sources of nitrogen and sulfur which can be assimilated to support growth and other cellular functions. While some odor-causing compounds can be consumed by many types of microorganisms, others are more difficult to digest and require specialized metabolic pathways. As disclosed herein, two microbial strains have been identified that are able to digest a variety of difficult to digest odor-causing compounds. Bacillus pumilus Bp D10 (NRRL No. B-68443) and Bacillus subtilis Bs E12 (NRRL No. B-68444) are able to digest volatile fatty acids, nitrogen compounds and sulfur compounds.

Accordingly, the present disclosure relates to treatment of waste with one or more microorganisms for the purposes of, including but not limited to, degrading waste, bioremediation of waste, reducing odor in waste, reducing organics in waste, and combinations thereof. More particularly, the present disclosure relates to compositions comprising isolated Bacillus strains, and strains having all of the identifying characteristics of these strains, and combinations thereof, for use in treating waste to reduce odors associated with organic waste.

### SUMMARY

The present disclosure relates generally to microorganisms for use in treating waste to reduce the presence of various organic compounds in waste, and thus reduce odors associated with such waste. In one embodiment a composition comprising a Bacillus strain, selected from the group consisting of Bacillus pumilus Bp D10 (NRRL No. B-68443), and Bacillus subtilis Bs E12 (NRRL No. B-68444), is used as a means of metabolizing or otherwise degrading an odor-causing compound present in said waste. Applicant has discovered that the Bacillus strains can degrade odor-causing compounds in waste including volatile fatty acids (e.g., propionic acid, butyric acid, lactic acid, acetic acid and valeric acid), nitrogen containing compounds (e.g., uric acid, ammonia, trimethylamine skatole and putrescine), sulfur containing compounds (e.g. dimethyl sulfide, ethyl mercaptan and benzyl mercaptan), and the like.

In one embodiment a method of reducing odors from waste is provided, wherein the waste to be treated is selected from the group consisting of industrial waste, municipal solid waste, landfill waste, wastewater, composting waste, and leachate from waste. The method comprises contacting the waste with an effective amount of an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof, and metabolizing or otherwise degrading or eliminating an odor-causing compound present in said waste.

In another embodiment, a method of controlling a detrimental effect of waste produced by an odor-causing compound is provided. The method comprises contacting the waste with an effective amount of an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof, and controlling the detrimental effect of the waste. In one embodiment, controlling a detrimental effect comprises reducing organics in waste (e.g., hydrocarbons), and/or reducing odor in waste (e.g., reducing H₂S and sulfate concentrations),

In various other embodiments, an additive for treating waste in water is provided. The additive for waste, comprises an isolated Bacillus strain selected from the group consisting of Bacillus pumilus Bp D10 (NRRL NO. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof. Optionally, the additive further comprises an odor counteractant selected from the group consisting of a zinc-based odor neutralizer, a sulfur binder, and an odor-masking fragrance, and any combination thereof.

### BRIEF DESCRIPTION OF THE DRAWINGS

FIGS. 1A and 1B: Growth in control media and media containing uric acid, ammonia, trimethylamine, skatole, or putrescine as the primary nitrogen source was determined after inoculation with Bp D10 (FIG. 1A) or Bs E12 (FIG. 1B) after 48 hours. Asterisk indicates the value is significantly different (p<0.05) than the control. A greater amount of growth compared to control indicates the strain was able to utilize the compound as a source of nitrogen.

FIGS. 2A and 2B: Growth in control media and media containing lactic acid, acetic acid, valeric acid or acetic acid as the primary carbon source was determined after inoculation with Bp D10 (FIG. 2A) or Bs E12 (FIG. 2B) after 48 hours. Asterisk indicates the value is significantly different (p<0.05) than the control. A greater amount of growth compared to control indicates the strain was able to utilize the volatile fatty acid as a source of carbon.

FIGS. 3A and 3B: Growth in control media and media containing dimethyl sulfide, benzyl mercaptan, or ethyl mercaptan as the primary sulfur source was determined after inoculation with Bp D10 (FIG. 3A) or Bs E12 (FIG. 3B) after 48 hours. Asterisk indicates the value is significantly different (p<0.05) than the control. A greater amount of growth compared to control indicates the strain was able to utilize the compound as a source of sulfur.

FIGS. 4A-4F show photographs of gels displaying a randomly amplified polymorphic DNA PCR method (RAPD) PCR profiles (Primers 1-6) for Bacillus pumilus Bp D10 and Bacillus subtilis Bs E12.

FIG. 5 is a bar graph demonstrating the reduction in propionic acid in minimal media treated with a blend of Bs E12 and Bp D10 after 5 days compared to control. Asterisk indicates value is significantly different (p<0.05, Student's t-test) from control.

FIG. 6 is a bar graph demonstrating the reduction in butyric acid in minimal media treated with a blend of E12 and D10 after 5 days compared to control. Asterisk indicates value is significantly different (p<0.05, Student's t-test) from control.

FIG. 7 is a bar graph demonstrating the reduction in acetic acid in minimal media treated with a blend of E12 and D10 after 5 days compared to control. Asterisk indicates value is significantly different (p<0.05, Student's t-test) from control.

FIG. 8 is a bar graph demonstrating the reduction in ammonia in minimal media treated with a blend of E12 and D10 after 5 days compared to control. Asterisk indicates value is significantly different (p<0.05, Student's t-test) from control.

### DETAILED DESCRIPTION

### Definitions

As used herein the term “waste” includes any material or substance that is unwanted, unusable, or rejected after its primary use, and is considered worthless or of no further use. This includes but is not limited to industrial waste, municipal solid waste, landfill waste, wastewater, composting waste, and leachate from waste.

As used herein the term “industrial waste” defines any byproduct or material that becomes useless from manufacturing, mining, processing, or research and development activities, including solids, liquids, and gases.

As used herein the term “municipal solid waste” defines any non-hazardous refuse, including organic waste, and yard waste.

As used herein “organic waste” defines any biodegradable material derived from plant or animal sources that can be broken down by microorganisms.

As used herein the term “landfill waste” defines any waste that is disposed of by burying it in a landfill.

As used herein the term “wastewater” defines any water based composition that has been polluted by contaminants like organic matter, microorganisms, nutrients, and chemicals, making it unsuitable for direct reuse or release into the environment.

As used herein the term “leachate from waste” defines water that has percolated through waste and flows out of the waste contaminated with components present in the waste.

As used herein the term “composting waste” defines any organic waste that results from the efforts to facilitate the natural process of breaking down organic materials.

As used herein the term “odor-causing compound” defines any chemical compound responsible for generating an odor sensation, wherein “odor” is defined as any gaseous emanation perceptible through the sense of smell.

As used herein an “exogenous source of enzyme” defines purified enzyme that is added to a composition from a source that is external to the original composition. For example, a composition comprising Bacillus strains can be supplemented with purified enzymes wherein the purified enzymes are currently being expressed by the Bacillus strain, or alternatively the added purified enzymes may represent enzymes that are not produced by the Bacillus strain.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

The term “isolated” requires that the referenced material be removed from its original environment (e.g., the natural environment if it is naturally occurring). For example, a naturally-occurring microbe present in the wild is not isolated, but the same microbe, separated from some or all of the coexisting materials and microbes in the natural system, is isolated.

As used herein reference to decreasing, reducing, or eliminating odor-causing compounds encompasses completely removing the odor-causing compounds, reducing the amount of the odor-causing compounds, degrading the odor-causing compounds, or converting the odor-causing compound into a form that does not produce an odor.

As used herein “controlling a detrimental effect of waste” or similar phrases means completely removing a compound causing the detrimental effect, reducing the amount of the compound causing the detrimental effect, degrading the compound causing the detrimental effect, or causing the compound responsible for the detrimental effect to be converted to an inactivated form.

“Controlling a detrimental effect of waste” encompasses degrading waste, enhancing waste stabilization, reducing contaminants in waste, reducing COD, reducing organics in waste (e.g., hydrocarbons), reducing odor in waste (e.g., H₂S and sulfate), and the like.

As used herein the phrase “under conditions suitable for growth of the Bacillus strains” defines conditions where the temperature, pH, osmotic pressure, oxygen concentration, and the availability of nutrients that are acceptable for the viability of Bacillus strains.

### ILLUSTRATIVE EMBODIMENTS

Applicants have developed Bacillus strains, including Bp D10 and Bs E12, and combinations thereof, that are useful for waste treatment, waste degradation (including degradation of odor-causing compounds) and controlling the detrimental effects of waste, such as by degrading odor-causing compounds. The strains can also be used as a component in commercial and household cleaning products These strains can increase the rate of decay of odor-causing compounds (e.g., volatile fatty acids, nitrogen containing compounds, and/or sulfur containing compounds), degrade municipal solid waste, enhance waste stabilization, reduce contaminants in waste, reduce chemical oxygen demand, reduce organics in waste (e.g., hydrocarbons), reduce odor in waste (e.g., hydrogen sulfide and sulfate), and the like. More particularly, the disclosure relates to isolated Bacillus pumilus and Bacillus subtilis strains, and strains having all of the identifying characteristics of these strains, and combinations thereof, for uses comprising the above-mentioned uses. Bp D10 is a Bacillus pumilus strain and Bs E12 is a Bacillus subtilis strain.

Bacillus pumilus Bp D10 and Bacillus subtilis Bs E12 were deposited on Oct. 9, 2024, at the Agricultural Research Service Culture Collection (NRRL), International Depository Authority, 1815 North University Street, Peoria, Illinois 61604-3999, and were given accession numbers NRRL No. B-68443, and NRRL No. B-68444, respectively. The deposits were made under the provisions of the Budapest Treaty on the International Recognition of the Deposit of Microorganisms for the Purposes of Patent Procedure. Any and all restrictions imposed by Applicant on the availability to the public of the deposited material will be irrevocably removed upon the granting of the patent, and the deposits will be replaced if viable samples cannot be dispensed by the depository. The NRRL strain designations are Bp D10, and Bs E12, which are equivalent to Bacillus pumilus Bp D10, and Bacillus subtilis Bs E12, respectively, as referred to in the application.

In one embodiment a method of treating waste is provided that reduces or removes an odor-causing compound, or that modifies/metabolizes the odor-causing compound to reduce or eliminate odors associated with organic waste. The method comprises contacting the waste with an effective amount of an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof, and removing, or reducing the concentration, of odor-causing compounds. In one embodiment the waste is directly contacted with a composition comprising an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof, under conditions suitable for growth of Bacillus strains. In one embodiment the Bacillus comprising composition is in the form of a dry powder, or is in the form of a liquid that can be sprayed on the waste. In one embodiment the composition further comprises an odor counteractant selected from the group consisting of a zinc-based odor neutralizer, a sulfur binder, and an odor-masking fragrance, and any combination thereof.

In accordance with one embodiment a method of reducing the concentration of one or more target compounds in a composition of matter is provided wherein the target compounds represent volatile fatty acids, nitrogen compounds and sulfur compounds that are difficult to metabolize through bioremediation. In one embodiment the target compounds include compounds selected from the group consisting of propionic acid, butyric acid, acetic acid, lactic acid, valeric acid, ammonia, uric acid, trimethylamine, skatole, putriscine, or a sulfur containing compound, including for example, methyl sulfide, ethyl mercaptan and benzyl mercaptan.

In one embodiment the method of reducing the concentration of one or more target compounds in a composition of matter comprises contacting the composition of matter with a remediating composition comprising an effective amount of an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof under conditions suitable for growth of Bacillus strains. In one embodiment the method comprises contacting the composition of matter with a remediating composition comprising an effective amount of an isolated Bacillus strain selected from the group consisting of Bacillus pumilus Bp D10 (NRRL No. B-68443), and Bacillus subtilis Bs E12 (NRRL No. B-68444), and combinations thereof. In one embodiment the method comprises contacting the composition of matter with a remediating composition comprising an effective amount of an isolated Bacillus strain selected from the group consisting of Bacillus pumilus Bp D10 (NRRL No. B-68443), Bacillus subtilis Bs E12 (NRRL No. B-68444), and combinations thereof. In one embodiment the remediating composition comprises a mixture of Bacillus pumilus Bp D10 (NRRL No. B-68443) and Bacillus subtilis Bs E12 (NRRL No. B-68444). In one embodiment the remediating composition further comprises one or more additional components selected from: a non-ionic surfactant, an anionic surfactant, a cationic surfactant, a rhamnolipid biosurfactant, or any combination thereof, an odor counteractant selected from the group consisting of a zinc-based odor neutralizer, a sulfur binder, and an odor-masking fragrance, and any combination thereof, a binder, a preservative or a formulation stabilizer. In one embodiment the composition of matter comprises industrial waste, municipal solid waste, wastewater, composting waste, and leachate from waste.

In another embodiment, a method of controlling a detrimental effect of waste is provided. The method comprises contacting the waste with an effective amount of an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof, and controlling the detrimental effect of the waste.

In another embodiment, a cleaning product is provided that reduces or removes an odor-causing compound, or that modifies/metabolizes the odor-causing compound to reduce or eliminate odor. The cleaning product might be used as a surface cleaner, a floor cleaner, or a drain cleaner in both commercial and household applications. The cleaning product comprises an effective amount of an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof. Additional components of the cleaning product could contain, but are not limited to, enzymes, surfactants, odor counteractants, preservatives, and formulation stabilizers, and combinations thereof. In one embodiment the cleaning product comprises an odor counteractant selected from the group consisting of a zinc-based odor neutralizer, a sulfur binder, and an odor-masking fragrance, and any combination thereof, optionally in further combination with a surfactant.

In one embodiment a method is provided for preventing or inhibiting the accumulation of odor-causing compounds on a surface, said method comprising applying or coating a surface with a composition comprising an effective amount of an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof. In one embodiment the composition is applied as a liquid that forms a coating on the surface. Additional components can be added to the composition, including, but not limited to, enzymes, surfactants, odor counteractants, preservatives, and formulation stabilizers, and combinations thereof. In one embodiment the composition comprises an odor counteractant selected from the group consisting of a zinc-based odor neutralizer, a sulfur binder, and an odor-masking fragrance, and any combination thereof, optionally in further combination with a surfactant.

In various other embodiments, a commercial package, an additive for waste, and a composition are provided. The commercial package, additive for waste, and composition comprise an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof, optionally in combination with an odor counteractant selected from the group consisting of a zinc-based odor neutralizer, a sulfur binder, and an odor-masking fragrance, and any combination thereof, optionally further in combination with a surfactant.

The following exemplary embodiments, and combinations thereof, provide various additional illustrative aspects of the invention described herein.

In accordance with embodiment 1, a method of reducing odors from waste or contaminated surface is provided wherein the method comprises

contacting the waste or contaminated surface with an effective amount of an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444) and combinations thereof, under conditions suitable for growth of the Bacillus strains, and reducing an odor-causing compound present in said waste or on the contaminated surface, optionally via metabolism or degradation of the odor-causing compound by the Bacillus strain.

In accordance with embodiment 2, a method for reducing or removing an odor-causing compound from a composition of matter is provided, wherein the method comprises contacting the composition of matter with a composition comprising an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof, under conditions suitable for growth of the Bacillus strain, to metabolize or degrade the odor-causing compound

In accordance with embodiment 3 the method of embodiment 1 or 2 is provided, wherein the odor-causing compound is
i) a volatile fatty acid, optionally propionic acid, butyric acid, lactic acid, acetic acid, or valeric acid;
ii) a nitrogen containing compound, optionally uric acid, ammonia, trimethylamine, skatole, or putrescine;
iii) a sulfur containing compound, optionally a dimethyl sulfide, ethyl mercaptan or benzyl mercaptan; or
iv) any combination of i), ii) or iii), optionally wherein the composition of matter to be treated is waste comprising organic compounds.

In accordance with embodiment 4 the method of embodiment 1 is provided wherein the waste is selected from the group consisting of industrial waste, municipal solid waste, landfill waste, wastewater, composting waste, and leachate from waste.

In accordance with embodiment 5, a method is provided for preventing or inhibiting the accumulation of odor-causing compounds on a surface, said method comprising applying or coating a surface with a composition comprising an effective amount of an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof.

In accordance with embodiment 6 the method of embodiment 5 is provided wherein the composition is applied as a liquid that forms a coating on the surface.

In accordance with embodiment 7, the method of any one of embodiments 1-6 is provided wherein the contaminated surface is a surface on tile, grout, a drain, a waste pipe, or a waste disposal unit.

In accordance with embodiment 8, the method of any one of embodiments 1-7 is provided wherein the composition comprising the isolated Bacillus strain comprises a mixture of Bacillus pumilus Bp D10 (NRRL No. B-68443) and Bacillus subtilis Bs E12 (NRRL No. B-68444).

In accordance with embodiment 9 the method of any one of embodiments of embodiment 1-8 is provided wherein the odor-causing compound is selected from the group consisting of a volatile fatty acid, a nitrogen containing compound and a sulfur containing compound.

In accordance with embodiment 10 the method of embodiment 9 is provided wherein the volatile fatty acid is propionic acid, butyric acid, lactic acid, acetic acid or valeric acid.

In accordance with embodiment 11 the method of embodiment 9 is provided wherein the nitrogen containing compound is selected from the group consisting of uric acid, ammonia, trimethylamine skatole, and putrescine.

In accordance with embodiment 12 the method of embodiment 9 is provided wherein the sulfur containing compound is dimethyl sulfide, ethyl mercaptan or benzyl mercaptan.

In accordance with embodiment 13 the method of any one of embodiments 1 to 12 is provided wherein the odor-causing compound is removed by degradation.

In accordance with embodiment 14 the method of any one of embodiments 1 to 13 is provided wherein the odor-causing compound is an organic compound.

In accordance with embodiment 15 the method of any one of embodiments 1 to 14 is provided wherein the Bacillus strain produces an enzyme selected from the group consisting of a lipase, an amylase, a protease, a cellulase, a keratinase, and combinations thereof.

In accordance with embodiment 16 the method of embodiments 1 to 14 is provided wherein the waste, contaminated surface or composition of matter is further contacted with an isolated enzyme selected from a lipase, an amylase, a protease, or any combination thereof.

In accordance with embodiment 17 the method of any one of embodiments 1 to 7 or 9-16 is provided wherein the strain is Bacillus pumilus Bp D10 (NRRL No. B-68443), or Bacillus pumilus Bp D10 (NRRL No. B-68443).

In accordance with embodiment 18 the method of any one of embodiments 1 to 7 or 9-17 is provided wherein the strain is Bacillus pumilus Bp D10 (NRRL No. B-68443).

In accordance with embodiment 19 the method of any one of embodiments 1 to 7 of 9-17 is provided wherein the strain is Bacillus subtilis Bs E12 (NRRL No. B-68444).

In accordance with embodiment 20 the method of any one of embodiments 1 to 19 is provided wherein the effective amount of the Bacillus strain is about 1.0×10² CFU/gram of the waste to about 1.0×10⁹ CFU/gram of the waste, or CFU/mL of water.

In accordance with embodiment 21 the method of any one of embodiments 1 to 19 is provided wherein the effective amount of the Bacillus strain is about 1.0×10² CFU/g of the waste to about 1.0×10⁷ CFU/g of the waste, or CFU/mL of water.

In accordance with embodiment 22 the method of any one of embodiments 1 to 19 is provided wherein the effective amount is an amount greater than about 1.0×10² CFU/g of the waste to about 1.0×10¹⁰ CFU/g of the waste, or CFU/mL of water.

In accordance with embodiment 23 the method of any one of embodiments 1 to 22 is provided wherein the strain is in a form selected from the group consisting of a powder, a liquid, and a particulate form.

In accordance with embodiment 24 the method of any one of embodiments 1 to 22 is provided wherein the strain is formulated as a slow release wax-based block or puck, tablet, or briquette.

In accordance with embodiment 25 the method of any one of embodiments 1 to 22 is provided wherein the strain is formulated as a liquid, optionally in combination with a surfactant, an odor counteractant, a preservative and/or a stabilizer.

In accordance with embodiment 26 the method of embodiment 25 is provided wherein the surfactant is selected from the group consisting of non-ionic surfactants, anionic surfactants, cationic surfactants, and rhamnolipid biosurfactants.

In accordance with embodiment 27 the method of embodiment 25 is provided wherein the odor counteractant is selected from zinc-based odor neutralizers, sulfur binders, odor-masking fragrances and any combination thereof.

In accordance with embodiment 28, a method of controlling a detrimental effect of waste is provided, wherein the method comprises contacting the waste with an effective amount of an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof, and controlling the detrimental effect of the waste.

In accordance with embodiment 29, the method of embodiment 28 is provided wherein the waste is selected from the group consisting of industrial waste, municipal solid waste, landfill waste, wastewater, composting waste, contaminated groundwater, and leachate from waste.

In accordance with embodiment 30 the method of embodiment 28 or 26 is provided wherein the detrimental effect is caused by an odor-causing compound selected from the group consisting of a volatile fatty acid, a nitrogen containing compound and a sulfur containing compound.

In accordance with embodiment 31, the method of embodiment 30 is provided wherein the volatile fatty acid is lactic acid, acetic acid or valeric acid.

In accordance with embodiment 32, the method of embodiment 30 is provided wherein the nitrogen containing compound is selected from the group consisting of uric acid, ammonia, and trimethylamine.

In accordance with embodiment 33, the method of embodiment 30 is provided wherein the sulfur containing compound is dimethyl sulfide or benzyl mercaptan.

In accordance with embodiment 34, the method of any one of embodiments 28 to 33 is provided wherein the detrimental effect is caused by an organic compound.

In accordance with embodiment 35, the method of embodiment 34 is provided wherein the organic compound is removed by degradation.

In accordance with embodiment 36, the method of any one of embodiments 28 to 33 is provided wherein the detrimental effect is caused by an inorganic compound.

In accordance with embodiment 37, the method of any one of embodiments 28 to 36 is provided wherein the Bacillus strain produces an enzyme selected from the group consisting of a lipase, an amylase, a protease, a cellulase, a keratinase, and combinations thereof.

In accordance with embodiment 38, the method of any one of embodiments 28 to 37 is provided wherein the strain is Bacillus pumilus Bp D10 (NRRL No. B-68443), or Bacillus subtilis Bs E12 (NRRL No. B-68444).

In accordance with embodiment 39, the method of any one of embodiments 28 to 38 is provided wherein the waste is contacted with a composition comprising Bacillus pumilus strain Bp D10 (NRRL No. B-68444) and Bacillus subtilis Bs E12 (NRRL No. B-68444).

In accordance with embodiment 40, the method of any one of embodiments 28 to 38 is provided wherein the strain is Bacillus pumilus Bp D10 (NRRL No. B-68443).

In accordance with embodiment 41, the method of any one of embodiments 28 to 38 is provided wherein the strain is Bacillus subtilis Bs E12 (NRRL No. B-68444).

In accordance with embodiment 42, the method of any one of embodiments 28 to 41 is provided wherein the effective amount of the Bacillus strain is about 1.0×10² CFU/g of the waste to about 1.0×10⁹ CFU/g of the waste.

In accordance with embodiment 43, the method of any one of embodiments 28 to 41 is provided wherein the effective amount of the Bacillus strain is about 1.0×10² CFU/g of the waste to about 1.0×10¹⁰ CPU/g of the waste.

In accordance with embodiment 44, the method of any one of embodiments 28 to 43 is provided further comprising contacting the waste with an enzyme selected from the group consisting of a lipase, an amylase, a protease, a cellulase, a keratinase, and combinations thereof.

In accordance with embodiment 45, a commercial package is provided, comprising an isolated Bacillus strain selected from the group consisting of Bacillus pumilus Bp D10 (NRRL No. B-68443), a strain having all of the identifying characteristics of Bacillus pumilus Bp D10 (NRRL No. 68443), Bacillus subtilis Bs E12 (NRRL No. B-68444), a strain having all of the identifying characteristics of Bacillus subtilis Bs E12 (NRRL No. B-68444), and combinations thereof, optionally in combination with an odor counteractant selected from the group consisting of a zinc-based odor neutralizer, a sulfur binder, and an odor-masking fragrance, and any combination thereof, optionally further in combination with a surfactant.

In accordance with embodiment 46, the commercial package of embodiment 45 is provided wherein the Bacillus strain causes an effect selected from the group consisting of a reduction in odor and a reduction in chemical oxygen demand.

In accordance with embodiment 47, an additive for treating waste is provided comprising an isolated Bacillus strain selected from the group consisting of Bacillus pumilus Bp D10 (NRRL No. B-68443), a strain having all of the identifying characteristics of Bacillus pumilus Bp D10 (NRRL No. 68443), Bacillus subtilis Bs E12 (NRRL No. B-68444), a strain having all of the identifying characteristics of Bacillus subtilis Bs E12 (NRRL No. B-68444), and combinations thereof, optionally wherein said additive further comprises an odor counteractant selected from the group consisting of a zinc-based odor neutralizer, a sulfur binder, and an odor-masking fragrance, and any combination thereof, optionally further in combination with a surfactant.

In accordance with embodiment 48, a composition comprising an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof.

In accordance with embodiment 49, the commercial package, additive, or composition of any one of embodiments 45-48 is provided wherein the Bacillus strain causes degradation of an organic compound or removal of an inorganic compound present in waste.

In accordance with embodiment 50, the commercial package, additive, or composition of embodiment 45-49 is provided wherein the Bacillus strain is in the form of a concentrate.

In accordance with embodiment 51, the commercial package, additive, or composition of any one of embodiments 45-49 is provided wherein the Bacillus strain is in the form of a superconcentrate.

In accordance with embodiment 52, the commercial package, additive, or composition of any one of embodiments 45-51 to is provided wherein the Bacillus strain is in dry form.

In accordance with embodiment 53, the commercial package, additive, or composition of any one of embodiments 45-52 is provided wherein the Bacillus strain is in particulate form.

In accordance with embodiment 54, the commercial package, additive, or composition of any one of embodiments 45-51 is provided wherein the strain is in a form selected from the group consisting of a powder, a liquid, and a particulate form.

In accordance with embodiment 55, the commercial package, additive, or composition of any one of embodiments 45-54 is provided further comprising a carrier for the Bacillus strain.

In accordance with embodiment 56, the commercial package, additive, or composition of embodiment 55 is provided wherein the carrier is selected from the group consisting of salt, a dextrin, and combinations thereof.

In accordance with embodiment 57, the commercial package, additive, or composition of any one of embodiments 45-56 is provided further comprising a surfactant, an odor counteractant, a preservative and/or a stabilizer.

In accordance with embodiment 58 the commercial package, additive, or composition of embodiment 57 is provided wherein the surfactant is selected from the group consisting of non-ionic surfactants, anionic surfactants, cationic surfactants, and rhamnolipid biosurfactants.

In accordance with embodiment 59 the commercial package, additive, or composition of embodiment 57 is provided wherein the odor counteractant is selected from zinc-based odor neutralizers, sulfur binders, odor-masking fragrances and any combination thereof.

In accordance with embodiment 60, the commercial package, additive, or composition of any one of embodiments 45 to 59 is provided in a bag.

In accordance with embodiment 61, the commercial package, additive, or composition of embodiment 60 wherein the bag is a plastic bag.

In accordance with embodiment 62, the commercial package, additive, or composition of any one of embodiments 45 to 61 is provided, further comprising instructions for use of one or more of the Bacillus strains.

In accordance with embodiment 63, the commercial package, additive, or composition of any one of embodiments 45 to 60 is provided in a 20-pound bag.

In accordance with embodiment 64, the commercial package, additive, or composition of any one of embodiments 45 to 60 is provided in a 50-pound bag.

In accordance with embodiment 65, the commercial package, additive, or composition of any one of embodiments 45 to 64 is provided wherein the Bacillus strain is in powder form.

In accordance with embodiment 66, the commercial package, additive, or composition of any one of embodiments 45 to 64 is provided wherein the Bacillus strain is in liquid form, optionally further comprising a preservative and/or a stabilizer.

In accordance with embodiment 67, the method of any one embodiments 1 to 66 is provided wherein the detrimental effect is odor and the odor is prevented, reduced or eliminated.

In accordance with embodiment 68, the method of embodiment 67 is provided wherein the odor is caused by sulfate and hydrogen sulfide (H₂S) production.

In embodiment 69, a composition is provided comprising an effective amount of an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof, wherein the Bacillus strains are in a liquid, optionally in combination with an odor counteractant selected from the group consisting of a zinc-based odor neutralizer, a sulfur binder, and an odor-masking fragrance, and any combination thereof, optionally further in combination with a surfactant.

In accordance with embodiment 70, a composition of embodiment 69 is provided, further comprising an enzyme selected from the group consisting of a lipase, an amylase, a protease, a cellulase, and a keratinase, or any combination thereof.

In accordance with embodiment 71, a composition of embodiment 69 or 70 is provided further comprising a surfactant.

In accordance with embodiment 72, a composition of embodiment 71 is provided, wherein the surfactant is selected from the group consisting of a non-ionic surfactant, an anionic surfactant, a cationic surfactant, a rhamnolipid biosurfactant, or any combination thereof.

In accordance with embodiment 73, a composition of any one of embodiments 69-72 is provided, further comprising an odor counteractant.

In accordance with embodiment 74, a composition of embodiment 73 is provided, wherein the odor counteractant is selected from the group consisting of a zinc-based odor neutralizer, a sulfur binder, and an odor-masking fragrance, and any combination thereof.

In an embodiment 75, the composition of any one of embodiments 69-74 is provided, further comprising a preservative.

In an embodiment 76, the composition of any one of embodiments 69-75 is provided, further comprising a formulation stabilizer.

In various embodiments, the Bacillus strain used in accordance with the methods, commercial packages, additives for waste, and compositions described herein can be selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof.

Any of these strains can be used to treat waste alone or in combination, in the form of a commercial package, an additive for waste, or a composition as described herein (e.g., an additive or a composition further comprising a carrier and/or a binder). In one embodiment, multiple strains are used to treat waste in combination in a single composition. In another embodiment, multiple strains are used to treat waste in combination in separate compositions.

As used herein “a strain having all of the identifying characteristics of” Bacillus pumilus Bp D10, or Bacillus subtilis Bs E12 can be a mutant strain having all of the identifying characteristics of Bacillus pumilus Bp D10, or Bacillus subtilis Bs E12 (e.g., a DNA fingerprint based on DNA analysis that corresponds to the DNA fingerprint of Bacillus pumilus Bp D10, or Bacillus subtilis Bs E12, enzyme activities that correspond to Bacillus pumilus Bp D10, or Bacillus subtilis Bs E12, antimicrobial activity that corresponds to Bacillus pumilus Bp D10, or Bacillus subtilis Bs E12, antibiotic sensitivity and tolerance profiles that correspond to Bacillus pumilus Bp D10, or Bacillus subtilis Bs E12, or combinations thereof). In alternate embodiments, the mutation can be a natural mutation, or a genetically engineered mutation. In another embodiment, “a strain having all of the identifying characteristics of” Bacillus pumilus Bp D10, or Bacillus subtilis Bs E12 can be a strain, for example, produced by isolating one or more plasmids from Bacillus pumilus Bp D10, or Bacillus subtilis Bs E12 and introducing the one or more plasmids into another bacterium, such as another Bacillus strain, as long as the one or more plasmids contain DNA that provides the identifying characteristics of Bacillus pumilus Bp D10, or Bacillus subtilis Bs E12 (e.g., a DNA fingerprint based on DNA analysis that corresponds to the DNA fingerprint of Bacillus pumilus Bp D10, or Bacillus subtilis Bs E12).

In another embodiment, one or more of the Bacillus strains described in the preceding paragraphs (e.g., Bacillus pumilus Bp D10, or Bacillus subtilis Bs E12) can be used to treat waste or a contaminated surface along with another bacterial strain selected from the group consisting of another Bacillus strain. In still another embodiment, the additional Bacillus strain can be selected from the group consisting of Bacillus subtilis, Bacillus amyloliquefaciens, Bacillus licheniformis, Bacillus pumilus, Bacillus mojavensis, Bacillus methylotrophicus, other Bacillus strains, and combinations thereof. In yet another embodiment, one or more of the Bacillus strains described in the preceding paragraphs (e.g., Bacillus pumilus Bp D10, or Bacillus subtilis Bs E12) can be used to treat waste or a contaminated surface along with any other bacterial strain effective to treat waste to decrease or eliminate odor-causing compounds, or to control detrimental effects of waste. As used herein a contaminated surface is a surface that has one or more odor-causing compounds associated with the surface. The surface can be porous (e.g., carpet, fabric, sponge, sandstone, brick, concrete, grout, drywall, ceramic tiles) or non-porous (e.g., interior surface of drainage pipes, waste disposal systems, metal countertops).

The additive for waste, or the commercial product, described herein can be used to treat waste for any period of time that is effective to decrease or eliminate odor-causing compounds and/or control the detrimental effects of waste. For example, in one embodiment treatment of waste can occur daily. The time periods for treatment of waste are non-limiting and it should be appreciated that any time period or treatment schedule determined to be effective to decrease or eliminate odor-causing compounds and/or control the detrimental effects of waste may be used.

In various illustrative embodiments, the Bacillus strain (e.g., Bacillus pumilus Bp D10, and/or Bacillus subtilis Bs E12), or any other bacterial strains added in addition to Bacillus pumilus Bp D10, and/or Bacillus subtilis Bs E12, can be added to the waste at about 1.0×10² CFU/gram of the waste, optionally at about 1.0×10² CFU/gram of the waste to about 1.0×10³ CFU/gram of the waste, about 1.0×10² CFU/gram of the waste to about 1.0×10⁴ CFU/gram of the waste, about 1.0×10² CFU/gram of the waste to about 1.0×10⁵ CFU/gram of the waste, about 1.0×10² CFU/gram of the waste to about 1.0×10⁶ CFU/gram of the waste, about 1.0×10³ CFU/gram of the waste to about 5.0×10¹² CFU/gram of the waste or at about 1.0×10³ CFU/gram of the waste to about 1.0×10¹⁰ CFU/gram of the waste. In other embodiments, the Bacillus strain (e.g., Bacillus pumilus Bp D10, and/or Bacillus subtilis Bs E12) can be added to the waste at an amount greater than about 1.0×10² CFU/gram of the waste to about 1.0×10¹⁰, at about 1.0×10³ CFU/gram of the waste, at about 1.1×10³ CFU/gram of the waste, at about 1.25×10³ CFU/gram of the waste, at about 1.5×10³ CFU/pound of the waste, at about 1.75×10³ CFU/gram of the waste, at about 1.0×10⁴ CFU/gram of the waste, at about 2.0×10⁴ CFU/gram of the waste, at about 3.0×10⁴ CFU/gram of the waste, at about 4.0×10⁴ CFU/gram of the waste, at about 5.0×10⁴ CFU/gram of the waste, at about 6.0×10⁴ CFU/gram of the waste, at about 7.0×10⁴ CFU/gram of the waste, at about 8.0×10⁴ CFU/gram of the waste, at about 1.0×10⁵ CFU/gram of the waste, at about 1.0×10⁶ CFU/gram of the waste, at about 1.0×10⁷ CFU/gram of the waste, at about 1.0×10⁸ CFU/gram of the waste, at about 1.0×10⁹ CFU/gram of the waste, at about 1.0×10¹⁰ CFU/gram of the waste, at about 1.0×10¹¹ CFU/gram of the waste, or at about 1.0×10¹² CFU/gram of the waste.

In various illustrative embodiments, the Bacillus strain (e.g., Bacillus pumilus Bp D10, and/or Bacillus subtilis Bs E12), or any other bacterial strains added in addition to Bacillus pumilus Bp D10, and/or Bacillus subtilis Bs E12, can be added to the waste at about 1.0×10² CFU/mL of water, about 1.0×10² CFU/mL of water to about 1.0×10³ CFU/mL of water, about 1.0×10² CFU/mL of water to about 1.0×10⁴ CFU/mL of water, about 1.0×10² CFU/mL of water to about 1.0×10⁵ CFU/mL of water, about 1.0×10² CFU/mL of water to about 1.0×10⁶ CFU/mL of water, about 1.0×10³ CFU/mL of water to about 5.0×10¹² CFU/mL of water or at about 1.0×10³ CFU/mL of water to about 1.0×10¹⁰ CFU/mL of water.

In various embodiments, the waste described herein can be selected from the group consisting of industrial waste, municipal solid waste, landfill waste, wastewater, composting waste, contaminated groundwater, leachate from waste, hydrocarbon-containing waste, and odor-causing compounds. In another embodiment, the odor-causing compound is a compound selected from the group consisting of a volatile fatty acid, a nitrogen containing compound, and a sulfur containing compound.

In another embodiment where the waste is leachate from a landfill, the Bacillus strain can be added to the leachate from a landfill as a multiplier to increase dosage rates to the landfill when the leachate is applied back to the landfill (i.e., the strains multiply in the leachate so the dose that is applied back to the landfill is increased).

In various illustrative aspects, the odor-causing compounds to be removed from the waste can be selected from the group consisting of a harmful microorganism, a microorganism producing an odor-causing compound, an organic compound, an inorganic compound, a volatile fatty acid, a nitrogen containing compound, or a sulfur containing compound and combinations thereof, and the like.

In embodiments where the odor-causing compound is a volatile fatty acid, the volatile fatty acid can be removed by degradation. In this embodiment, the volatile fatty acid can be, for example, a propionic acid, butyric acid, lactic acid, acetic acid, or a valeric acid.

In embodiments where the odor-causing compound is a nitrogen containing compound, the nitrogen containing compound can be removed by degradation. In this embodiment, the nitrogen containing compound can be, for example uric acid, ammonia, trimethylamine, skatole, or putrescine.

In embodiments where the odor-causing compound is a sulfur containing compound, the sulfur containing compound can be removed by degradation. In this embodiment, the sulfur containing compound can be, for example, a dimethyl sulfide, ethyl mercaptan or benzyl mercaptan.

In one embodiment a method for treating organic waste to reduce or remove an odor-causing compound is provided. In one embodiment the method comprises contacting the organic waste with a composition comprising an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof, under conditions suitable for growth of the Bacillus strain, to metabolize or degrade the odor-causing compound, wherein the odor-causing compound is
i) a volatile fatty acid, optionally propionic acid, butyric acid, lactic acid, acetic acid, or valeric acid;
ii) a nitrogen containing compound, optionally uric acid, ammonia, trimethylamine, skatole, or putriscine;
iii) a sulfur containing compound, optionally a dimethyl sulfide, ethyl mercaptan or benzyl mercaptan; or
iv) any combination of i), ii) or iii).

In various illustrative aspects, the Bacillus strains described herein (i.e., Bacillus pumilus Bp D10, and/or Bacillus subtilis Bs E12) produce an enzyme selected from the group consisting of a lipase, an amylase, a protease, a cellulase, a keratinase, and combinations thereof.

In one illustrative embodiment, one or more enzymes may be added to the additive for waste, or the composition described herein, or may be added directly to the waste in combination with the Bacillus strains described herein. In various embodiments, the enzymes that may be used to treat the waste in addition to the Bacillus strains include a lipase, an amylase, a protease, a cellulase, a keratinase, and combinations thereof, and any other enzyme that is suitable to treat waste to remove odor-causing compounds or control a detrimental effect of waste. Any of the enzymes described above that are suitable for treatment of waste may be added as a component of the commercial package, additive for waste, or composition described herein, or may be added directly the waste as a separate composition.

In additional embodiments of the disclosure, compositions comprising Bacillus pumilus Bp D10, and/or Bacillus subtilis Bs E12 are provided. In one embodiment, a commercial package is provided comprising an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof.

In another embodiment, an additive for waste is provided comprising an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof.

In yet another embodiment, a composition is provided comprising an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof.

In these embodiments the Bacillus strain can be in the form of, for example, a powder, a liquid, or particulates, and can be mixed with the waste using any suitable method known in the art to achieve any of the amounts of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), or a strain having all of the identifying characteristics of Bacillus subtilis Bs E12 (NRRL No. B-68444), and combinations thereof, for the treatment of waste to remove, or reduce the concentration of odor-causing compounds or to control a detrimental effect of waste.

In these embodiments, the Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), or a strain having all of the identifying characteristics of Bacillus subtilis Bs E12 (NRRL No. B-68444), and combinations thereof, can cause degradation or removal of an organic compound, or degradation or removal of an inorganic compound in waste.

In illustrative aspects, the Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), or a strain having all of the identifying characteristics of Bacillus subtilis Bs E12 (NRRL No. B-68444), and combinations thereof, can be in the form of a commercial package, an additive for waste or any suitable composition. In another illustrative embodiment, the Bacillus strain(s) in the commercial package, additive for waste, or composition can be in the form of a concentrate (e.g., about 1×10⁸ to about 5×10⁹ CFU/g, or CFU/mL of water) or a superconcentrate (e.g., about 1×10¹⁰ to about 5×10¹² CFU/g, or CFU/mL of water). In another embodiment, the Bacillus strain(s) in the commercial package, additive for waste, or composition can be in a dry form (e.g., a powder), a particulate form, a liquid form, a freeze-dried form, or in the form of a gel, or any other suitable form. In one embodiment the Bacillus strain(s) in the commercial package, additive for waste, or composition can be formulated as a slow release formulation using a wax-based block or puck, tablet or briquette. In one embodiment the commercial package, additive for waste, or composition comprises a Bacillus strain of the present disclosure wherein the commercial package, additive for waste, or composition is formulated as a dry form (e.g., a powder), a pelleted form, a freeze-dried form, or in the form of a gel. In one embodiment the commercial package, additive for waste, or composition can be formulated as a liquid, optionally in combination with a surfactant, preservative or other component to minimize odors from waste.

In another illustrative embodiment, the Bacillus strain(s) in the commercial package, additive for waste, or composition can further comprise a carrier for the Bacillus strain(s). In various embodiments, the carrier can be selected from the group consisting of a salt, mineral oil, a dextrin (e.g., maltodextrin), whey, sugar, limestone, dried starch, sodium silico aluminate, and combinations thereof. In another embodiment, the carrier can be any suitable carrier known in the art for a composition for treating waste.

In yet other embodiments, the commercial package, additive for waste, or composition comprising Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), or a strain having all of the identifying characteristics of Bacillus subtilis Bs E12 (NRRL No. B-6844), and combinations thereof, is in a container for commercial use. In various embodiments the container can be, for example, a bag (e.g., a 20-pound bag, a 50-pound bag, a 2-ounce bag, a 1-pound bag, or a 1-kilogram bag), a pouch, a drum, a bottle, or a box. In illustrative aspects, the container comprising the Bacillus strain(s) can comprise plastic, metal, foil, paper, fiber, or cardboard (e.g., a plastic pail, a paper bag, a foil bag, a fiber drum, etc.). The commercial package can further comprise instructions for use of one or more of the Bacillus strains.

The following examples are for illustrative purposes only. The examples are non-limiting, and are not intended to limit the invention in any way.

### Example 1

### Bacillus Strains Bp D10 or Bs E12 Degrade Nitrogen Based Compounds

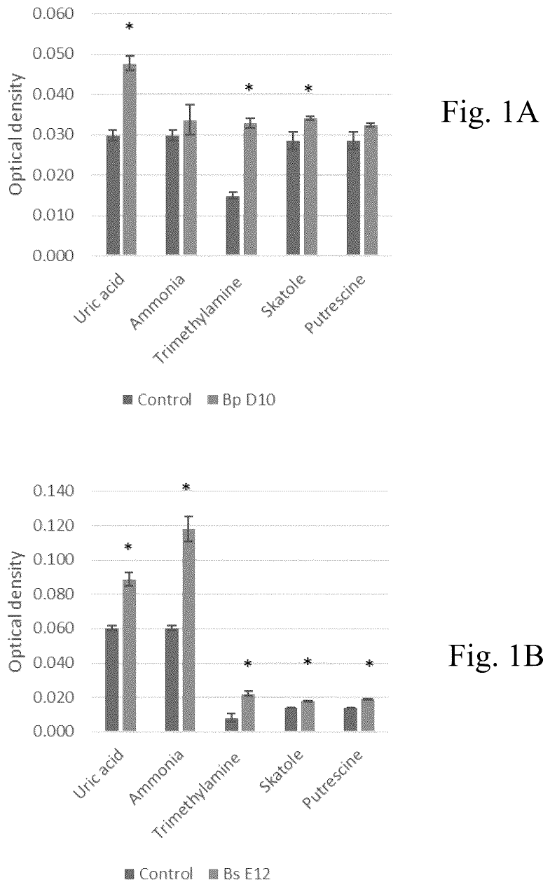

Bacillus strains Bp D10 and Bs E12 were tested for their ability to digest the nitrogen compounds uric acid, ammonia, trimethylamine, skatole, and putrescine. Sterile media was prepared with the compounds being the primary source of nitrogen and control media was prepared without the compound. Medias were inoculated with Bp D10 or Bs E12 and incubated at 25° C. for 48 hours. Bacterial growth was quantified by measuring optical density. An increase in growth compared to the control indicates that the strain is able to utilize the compound to support additional biomass growth. As shown in FIGS. 1A and 1B, Bacillus strains Bp D10 and Bs E12 were able to utilize the nitrogen compounds uric acid, ammonia, trimethylamine, skatole, and putrescine as a source of carbon, and thus were able to metabolize and remove such compounds from the media.

### Example 2

### Bacillus Strains Bp D10 or Bs E12 Degrade Volatile Fatty Acids

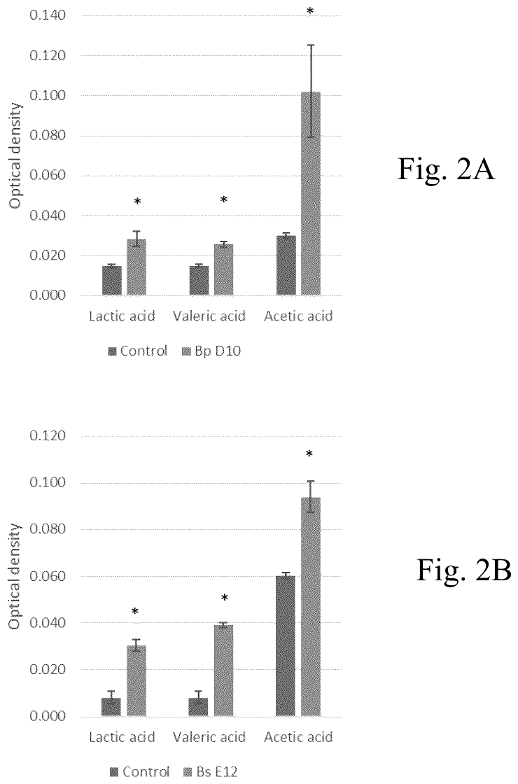

Bacillus strains Bp D10 and Bs E12 were tested for their ability to digest the volatile fatty acids (VFA) lactic acid, valeric acid, and acetic acid. Sterile media was prepared with the VFA being the primary source of carbon and control media was prepared without the VFA. Medias were inoculated with Bp D10 or Bs E12 and incubated at 25° C. for 48 hours. Bacterial growth was quantified by measuring optical density. An increase in growth compared to the control indicates that the strain is able to utilize the VFA to support additional biomass growth. As shown in FIGS. 2A and 2B, Bacillus strains Bp D10 and Bs E12 were able to utilize the volatile fatty acids (VFA) lactic acid, valeric acid, and acetic acid as a source of carbon, and thus were able to metabolize and remove such compounds from the media.

### Example 3

### Bacillus Strains Bp D10 or Bs E12 Degrade Sulfur Containing Compounds

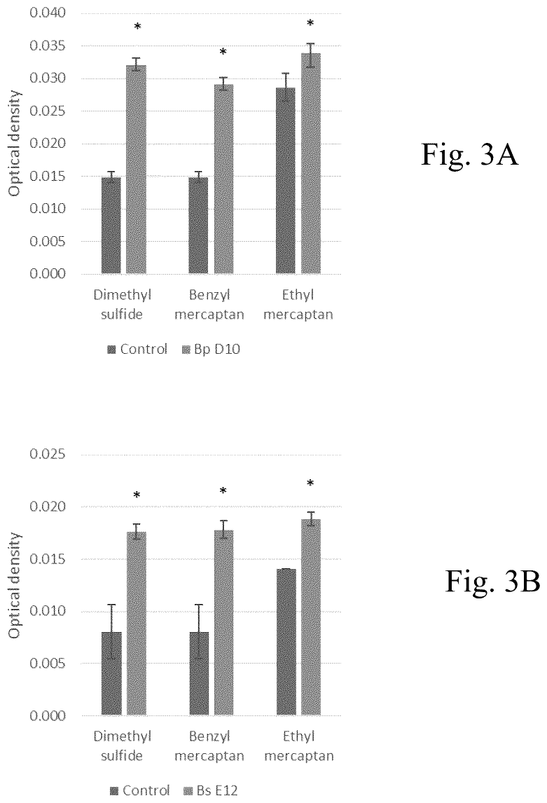

Bacillus strains Bp D10 and Bs E12 were tested for their ability to digest the sulfur containing compounds, dimethyl sulfide, benzyl mercaptan, and ethyl mercaptan. Sterile media was prepared with the VFA being the primary source of carbon and control media was prepared without the VFA. Medias were inoculated with Bp D10 or Bs E12 and incubated at 25° C. for 48 hours. Bacterial growth was quantified by measuring optical density. An increase in growth compared to the control indicates that the strain is able to utilize the VFA to support additional biomass growth. As shown in FIGS. 3A and 3B, Bacillus strains Bp D10 and Bs E12 were able to utilize the sulfur containing compounds, dimethyl sulfide, benzyl mercaptan, and ethyl mercaptan as a source of carbon, and thus were able to metabolize and remove such compounds from the media.

### Example 4

### Strain Identification and Uniqueness

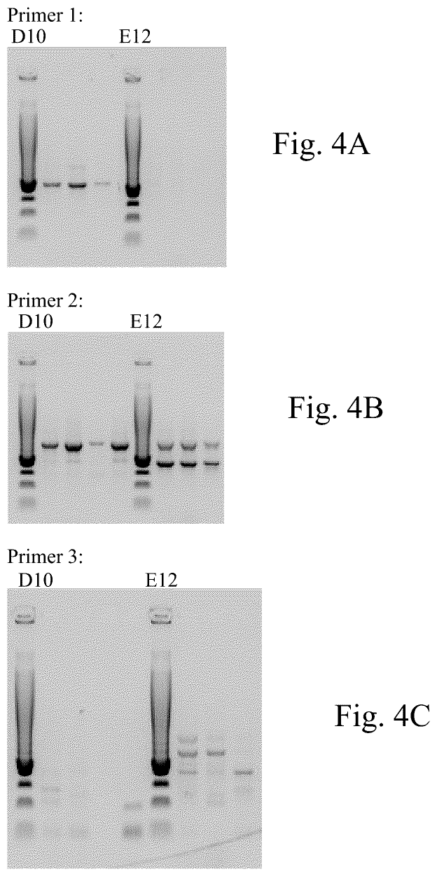

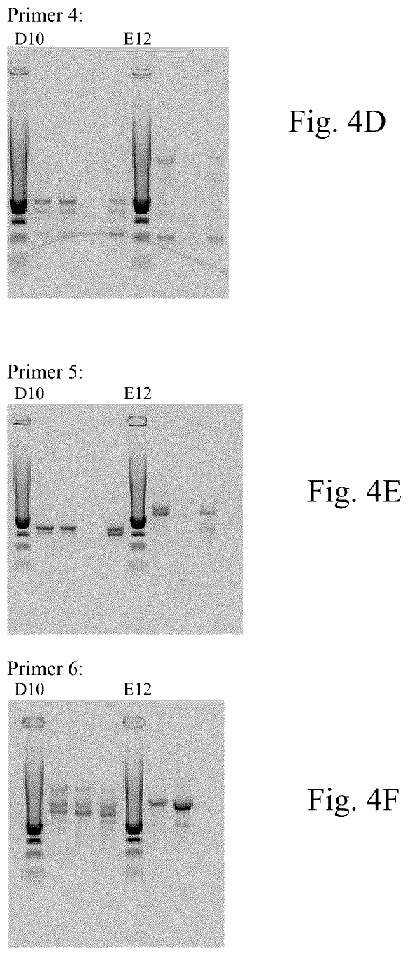

The Randomly Amplified Polymorphic DNA PCR method (hereafter referred to as RAPD-PCR) was used to identify genetic variability of each strain. Preparation of the DNA to be used in the RAPD-PCR reaction was done by using the QIAGEN® Tissue and Blood single column kit (QIAGEN®, Venlo, The Netherlands). FIGS. 4A-4F illustrate RAPD-PCR results for strains Bacillus strains Bp D10 and Bs E12. The results show that the two strains are unique from each other.

### Example 5

### Bacillus Strains Bp D10 or Bs E12 Degrade Propionic Acid, Butyric Acid, Acetic Acid, and Ammonia

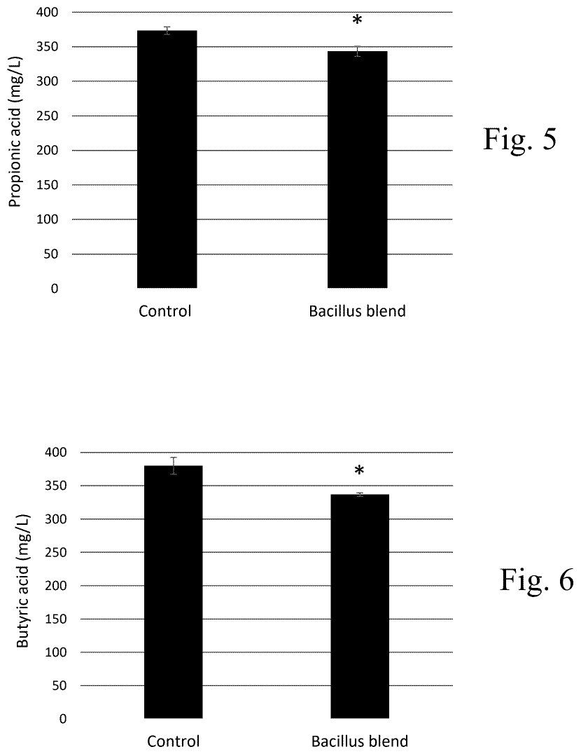

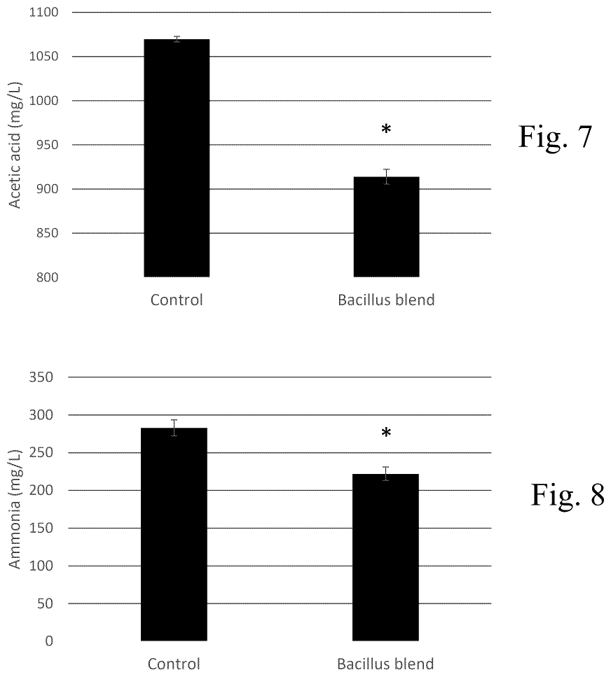

Reduction of propionic acid, butyric acid, acetic acid, and ammonia were evaluated after treatment with a blend of Bs E12 (NRRL #B-68444) and Bp D10 (NRRL #B-68443). Minimal media with ammonia, acetic acid, propionic acid, or butyric acid were left untreated (control) or treated with 1×10⁶ CFU/ml of the Bacillus blend for five days at 25° C. and 130 rpm. After treatment, butyric acid and propionic acid were quantified via GC-MS and ammonia and acetic acid were quantified using Hach kits Ammonia TNT 834 and Volatile Acids TNT 872, respectively. There was a significant reduction in all four odor compounds as a result of treatment with the Bacillus blend (p<0.05, Student's t-test) as shown in FIGS. 5-8.

1. A commercial package comprising an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof, wherein the Bacillus strains are in the form of a powder or a freeze-dried composition.

2. The commercial package of claim 1 wherein the package further comprises
i) and exogenous source of enzymes selected from the group consisting of a lipase, amylase, protease, cellulase and keratinase; or
ii) a surfactant.

3. The commercial package of claim 2 wherein the package further comprises said exogenous source of enzymes at a dosage of about 10² to 10¹ CFU/g of waste or CFU/ml of water.

4. The commercial package of claim 1 wherein the package further comprises an odor counteractant selected from the group consisting of a zinc-based odor neutralizer, a sulfur binder, an odor-masking fragrance and combinations thereof.

5. An additive for waste comprising
an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof, and
an odor counteractant selected from the group consisting of a zinc-based odor neutralizer, a sulfur binder, an odor-masking fragrance and combinations thereof.

6. The additive of claim 5 wherein the Bacillus strain is in a form selected from the group consisting of a powder, a liquid, particulate or a freeze-dried composition.

7. The additive of claim 5 further comprising a surfactant selected from the group consisting of a non-ionic surfactant, an anionic surfactant, a cationic surfactant, a rhamnolipid biosurfactant, or any combination thereof.

8. The additive of claim 5 further comprising a preservative.

9. The additive of claim 5 further comprising a formulation stabilizer.

10. A method of reducing the concentration of one or more target compounds in a composition of matter,
said target compounds including compounds selected from the group consisting of propionic acid, butyric acid, acetic acid, lactic acid, valeric acid and ammonia,
said method comprising contacting the composition of matter with a remediating composition comprising an effective amount of an isolated Bacillus strain selected from the group consisting of Bacillus pumilus Bp D10 (NRRL No. B-68443), a strain having all of the identifying characteristics of Bacillus pumilus Bp D10 (NRRL No. B-68443), Bacillus subtilis Bs E12 (NRRL No. B-68444), a strain having all of the identifying characteristics of Bacillus subtilis Bs E12 (NRRL No. B-68444), and combinations thereof.

11. The method of claim 10 wherein the composition of matter comprises industrial waste, municipal solid waste, wastewater, composting waste, and leachate from waste.

12. The method of claim 10 wherein the remediating composition comprises a mixture of Bacillus pumilus Bp D10 (NRRL No. B-68443) and Bacillus subtilis Bs E12 (NRRL No. B-68444).

13. The method of claim 10 wherein an odor-causing compound present in said composition of matter is metabolized or degraded to reduce reducing odors emanating from said composition.

14. The method of claim 10 wherein the odor-causing compound is selected from
i) a volatile fatty acid selected from the group consisting of propionic acid, butyric acid, lactic acid, acetic acid and valeric acid; or
ii) a nitrogen containing compound selected from the group consisting of uric acid, ammonia, trimethylamine, skatole, and putriscine; or
iii) a sulfur containing compound selected from the group consisting of methyl sulfide, ethyl mercaptan and benzyl mercaptan; and
iv) any combination of i)-iii).

15. The method of claim 10 wherein the Bacillus strain produces an enzyme selected from the group consisting of a lipase, an amylase, a protease, a cellulase, a keratinase, and combinations thereof.

16. The method of claim 10 wherein the Bacillus strain is Bacillus pumilus Bp D10 (NRRL No. B-68443).

17. The method of claim 10 wherein the Bacillus strain is Bacillus subtilis Bs E12 (NRRL No. B-68444).

18. The method of claim 13 wherein the remediating composition comprises a combination of Bacillus pumilus Bp D10 (NRRL No. B-68443) and Bacillus subtilis Bs E12 (NRRL No. B-68444).

19. The method of claim 10 wherein the effective amount of the Bacillus strain is about 1.0×10² CFU/g of the waste to about 1.0×10⁷ CFU/g of the waste, or is an amount greater than about 1.0×10² CFU/g of the waste to about 1.0×10¹⁰ CFU/g of the waste.

20. The method of claim 10 further comprising contacting the waste with an exogenous source of enzyme, wherein the enzyme is selected from the group consisting of a lipase, an amylase, a protease, a cellulase, and a keratinase, and combinations thereof.
